(19) United States
(12) Patent Application Publication
(10) Pub. No.: US 2026/0306533 A1
(43) Pub. Date: Oct. 8, 2026

(54) AMORPHOUS CALCIUM CARBONATE FOR PREVENTION AND TREATMENT OF MYOCARDIAL ISCHEMIA

(71) Applicant: Amorphical Ltd., Ness Ziona (IL)

(72) Inventors: Yossi BEN, Moshav Zofer (IL); Yigal Dov BLUM, San Jose, CA (US)

(21) Appl. No.: 19/485,460

(22) PCT Filed: May 28, 2024
(86) PCT No.: PCT/IL2024/050523

§ 371 Date: Nov. 18, 2025

Related U.S. Application Data
(60) Provisional application No. 63/504,795, filed on May 30, 2023.

(51) Int. Cl. A61K 33/10; A61P 9/10

(52) U.S. Cl. CPC A61K 33/10; A61P 9/10

(57) ABSTRACT

The present invention provides methods for treatment and/or prevention of myocardial ischemia and myocardial infarction comprising administering compositions comprising stabilized amorphous calcium carbonate.

### FIELD OF THE INVENTION

The present invention provides methods of treating or preventing myocardial ischemia by administering to a subject suffering from or predisposed to development of the myocardial ischemia stabilized amorphous calcium carbonate.

### BACKGROUND OF THE INVENTION

Myocardial ischemia refers to a kind of pathological state in which reduction of blood perfusion in heart causes reduced oxygen supply to heart, irregular energy metabolism of myocardium and abnormal work of heart. Clinical studies revealed that coronary artery stenosis, whose primary cause is arteriosclerosis, is the major and most common pathogenesis of myocardial ischemia. Coronary disease, what people often say heart disease, is caused by coronary atherosclerosis. At present, there are a lot of therapeutic regimes for myocardial ischemia which is a kind of coronary heart disease, such as stent placement and balloon dilation, whereas drug therapy is still a main method. Nitrate esters medicines (e.g. isosorbide mononitrate or its slow-release), beta blockers like propranolol and calcium channel blockers like nifedipine could be selected to dilate coronary arteries, increase myocardial oxygen supply, reduce peripheral resistance, working of heart and oxygen consumption of myocardium. Statins (e.g. atorvastatin, simvastatin) also should be taken to reduce plasma cholesterol and steady atherosclerotic plaque, preventing plaque from coming off to form thrombosis which causes stroke.

Myocardial infarction is myocardial necrosis due to persistent ischemia of partial myocardium, which results from atheromatous plaque bleeding and intravascular thrombogenesis caused from coronary atherosclerosis, which can result in rapidly, permanently and completely blocking of lumen of the blood vessel, and discontinuing of the bloodstream. It can be divided into 3 phases according to clinical process and ECG manifestation, which are acute, subacute and chronic phases. The clinical symptoms mainly occur in the acute phase, and the highest mortality is in the first week of the acute phase. Acute myocardial infarction (AMI) is a common severe case that threatens human life, and it is also currently one of the most harmful heart conditions.

WO 2008/041236 describes treating musculoskeletal disorders by compositions comprising calcium carbonate (CaCO₃) finely mixed with an organic matter essentially consisting of chitin and polypeptide (Pp), wherein said specific ratios between CaCO₃ and organic matter or said polypeptide is indicated.

WO 2013/088440 disclosed that the bioavailability of amorphous calcium carbonate was significantly higher than the bioavailability of crystalline calcium carbonate. Meiron (Journal of Bone and Mineral Research, Vol. 26, No. 2, 2011, pp 364-372) reached a similar observation and further stated that the amorphous calcium carbonate is approximately 120 times more soluble than calcite (one of the crystalline forms of calcium carbonate).

Seung-Kwon Myung et al., (Nutrients. 2021 Feb; 13(2): 368) and many other publications describe the devastating role of administering calcium supplements in people having cardiovascular diseases There is an unmet need for developing novel and effective means for treating and preventing myocardial ischemia.

### SUMMARY OF THE INVENTION

The present invention is based on the surprising finding that the administration of calcium carbonate, which is considered as undesired supplement in case of cardiovascular diseases provides a significant improvement in subjects suffering from myocardial ischemia and specifically from myocardial infarction.

According to one aspect, the present invention provides a composition comprising an amorphous calcium carbonate (ACC) stabilized by at least one stabilizer for use treatment or prevention of myocardial ischemia. According to some embodiments, the myocardial ischemia is caused by myocardial infarction. According to some embodiments, the myocardial ischemia is acute. According to some embodiments, the myocardial ischemia is chronic.

According to some embodiments, the composition is administered in a mode selected from sublingual, inhalation, IV, and oral administration. According to some embodiments, the composition is administered in combinatory administration mode selected from oral and sublingual, inhalation and sublingual, oral and inhalation and oral, inhalation and sublingual administrations, and inhalation and with IV.

According to some embodiments, the composition is administered in a dose of from about 5 to about 200 mg/kg/day of stabilized ACC.

According to some embodiments, for sublingual and/or inhalation administration is a powder composition comprising secondary particles of ACC having a size of below 500 μm.

According to some embodiments, the stabilizer is selected from the group consisting of polyphosphates, organic acids, phosphorylated amino acids, phosphorylated, phosphonated, sulfated or sulfonated organic compounds, phosphoric or sulfuric esters of hydroxy carboxylic acids, bisphosphonates, organic polyphosphates, polyphosphates, hydroxyl bearing organic compounds, derivatives thereof, proteins and any combinations thereof.

According to some embodiments, the stabilizer is selected from the group consisting of triphosphate or a salt thereof, phosphoserine, citric acid, sodium triphosphate and citric acid, adenosine triphosphate, adenosine diphosphate, phytic acid, etidronic acid, pyrophosphate, polyphosphate, hexamethaphosphate, ethanol, and any combination thereof.

According to another aspect, the present invention provides a method for treating or preventing myocardial ischemia in a subject comprising administering to said subject a composition comprising amorphous calcium carbonate (ACC) stabilized by at least one stabilizer.

According to any one of the above embodiments, the composition is formulated as a food supplement or as a pharmaceutical composition that may be in the form of powder, suspension, tablets, or capsules.

### BRIEF DESCRIPTION OF THE DRAWINGS

FIG. 1 shows the results of echocardiograph of 3 treatment groups on days 0, 2, and 28 (One-way ANOVA P=0.0120).

FIG. 2 shows the percentage of the change in the Ejection Fraction of each treatment group on day 28 compared to baseline (day 0).

FIG. 3A shows the percentage of proliferating cardiomyocytes cultured with 1 or 2 mM ACC or CaCl₂. FIG. 3B shows proliferation of cardiomyocytes in the presence of ACC relative to proliferation in the presence of CaCl₂ (same concentration of elemental calcium). FIG. 3C shows mRNA expression of cTNT in the presence of ACC relative to expression in the presence of CaCl₂ (same concentration of elemental calcium).

FIG. 4 shows mRNA levels of CD31 (endothelial marker) in cells cultured with 2 mM ACC (two repetitions) or CaCl₂.

FIG. 5 shows the ratio of new blood vessels compared to the damaged area in the 2 treatment groups (ACC and control) (p<0.05).

### DETAILED DESCRIPTION OF THE INVENTION

According to one aspect, the present invention provides a method for treating or preventing myocardial ischemia in a subject comprising administering to said subject a composition comprising amorphous calcium carbonate (ACC) stabilized by at least one stabilizer

According to another aspect, the present invention provides a composition comprising an amorphous calcium carbonate (ACC) stabilized by at least one stabilizer for use in the treatment or prevention of a myocardial ischemia. According to some embodiments, the present invention provides a composition comprising an amorphous calcium carbonate (ACC) stabilized by at least one stabilizer for use in the treatment of a myocardial ischemia. According to some embodiments, the present invention provides a composition comprising an amorphous calcium carbonate (ACC) stabilized by at least one stabilizer for use in prevention of a myocardial ischemia.

The terms “subject” or “individual” are used interchangeably and refer to either a human or a non-human animal. These terms include mammals, such as humans, primates, livestock animals (including bovine, porcine, etc.), companion animals (e.g., canines, felines, etc.) and rodents (e.g., mice and rats). According to other embodiments, the subject is an animal such as livestock animals or domestic animals. According to some embodiments, the subject is a human subject.

As used herein, the terms “myocardial ischemia” and “cardiac ischemia” is defined as an insufficient blood supply to the heart muscle caused by a decreased capacity of the heart vessels. The myocardial ischemia may be caused by a coronary disease. As used herein, the term “coronary disease” is defined as diseases/disorders of cardiac function due to an imbalance between myocardial function and the capacity of coronary vessels to supply sufficient blood flow for normal function. Specific coronary diseases/disorders associated with coronary disease which can be treated with the compositions and methods described herein include myocardial ischemia, angina pectoris, coronary aneurysm, coronary thrombosis, coronary vasospasm, coronary artery disease, coronary heart disease, coronary occlusion and coronary stenosis. An alternative cause of myocardial ischemia is caused by cardiac trauma. According to some embodiments, the myocardial ischemia is caused by atherosclerosis, blood clot or coronary artery spasm.

As used herein the term “occlusive peripheral vascular disease” (also known as peripheral arterial occlusive disorder) is a vascular disorder-involving blockage in the carotid or femoral arteries, including the iliac artery. Occlusive vascular diseases include but not limited to conditions in Sickle cell patients. Blockage in the femoral arteries causes pain and restricted movement. A specific disorder associated with occlusive peripheral vascular disease is diabetic foot, which affects diabetic patients, often resulting in amputation of the foot.

According to some embodiments, myocardial ischemia comprises myocardial infarction. Therefore, according to some embodiments, the present invention provides a composition comprising an amorphous calcium carbonate (ACC) stabilized by at least one stabilizer for use in the treatment or prevention of myocardial infarction.

The terms “treating” and “treatment” are uses herein interchangeably and refer to taking steps to obtain beneficial or desired results, including clinical results. Beneficial or desired clinical results include, but are not limited to, alleviation or amelioration of one or more symptoms associated with myocardial ischemia and in myocardial infarction, delay or slowing the development or the progression of ischemia, amelioration, palliation or stabilization of the disease, and other beneficial results. In particular, according to one embodiment, treating an ischemia comprises at least one of the following: ceasing, retarding reversing and preventing the development of the ischemia. According to some embodiments, treating or preventing myocardial ischemia or infarction comprises enhancing vascularization, i.e. angiogenesis. As used herein, the term “preventing” when used in relation to a condition or disease, refers to the administration of a composition which reduces the frequency of, the probability of, or delays the onset of, symptoms of a medical condition in a subject relative to a subject which does not receive the composition. Therefore, the preventing myocardial ischemia or infarction comprises administering the stabilized ACC to a subject predisposed or being in risk of suffering from myocardial ischemia or infarction. Such subjects are those who e.g. suffers from diabetes, high blood pressure, high blood cholesterol level, high blood triglyceride level, obesity, having genetic predisposition, stress, has a history of heart disease of MI or smoking subjects. Therefore, according to some embodiments, the present invention provides an amorphous calcium carbonate (ACC) stabilized by at least one stabilizer for use in the treatment or prevention of a myocardial ischemia is a subject suffering from diabetes. According to another embodiment, the present invention provides an amorphous calcium carbonate (ACC) stabilized by at least one stabilizer for use in the treatment or prevention of a myocardial ischemia is a subject suffering from high blood pressure. According to another embodiment, the present invention provides an amorphous calcium carbonate (ACC) stabilized by at least one stabilizer for use in the treatment or prevention of a myocardial ischemia is a subject suffering from high blood cholesterol level. According to another embodiment, the present invention provides an amorphous calcium carbonate (ACC) stabilized by at least one stabilizer for use in the treatment or prevention of a myocardial ischemia is a subject suffering from high blood triglyceride level.

The term “administering” or “administration of” a substance, a compound or a composition to a subject can be carried out using one of a variety of methods known to those skilled in the art. For example, a compound or a composition can be administered enterally or parenterally. Enterally refers to administration via the gastrointestinal tract including per os, or rectally. Parenteral administration includes administration intravenously, sublingually, intradermally, intramuscularly, intraperitoneally, subcutaneously, ocularly, sublingually, intranasally, by inhalation, intraspinally, intracerebrally, and transdermally (by absorption, e.g., through a skin duct). A compound or a composition can also appropriately be introduced by rechargeable or biodegradable polymeric devices or other “drug-release” and “controlled-release” devices, e.g., patches and pumps, or formulations, which provide for the extended, slow or controlled release of the compound or composition. Administering can also be performed, for example, once, a plurality of times, and/or over one or more extended periods. In some aspects, the administration includes both direct administration, including self-administration, and indirect administration, including the act of prescribing a drug or medical food.

According to some embodiments, the administration is an oral administration. According to other embodiments, the administration is a sublingual administration. According to further embodiments, the administration is a combined oral and sublingual administration. According to other embodiments, the administration is administration by inhalation. According to other embodiments, the administration is a combination of administration by inhalation and oral and/or sublingual administration. According to some embodiments, the composition is administered in a mode selected from sublingual, inhalation, IV, oral administration or by a combination of administration selected from oral and sublingual, inhalation and sublingual, oral and inhalation and oral, inhalation and sublingual administrations.

According to certain embodiments, administration, e.g. oral, sublingual, or combined, comprises administering less than 200 mg/kg per day of calcium as stabilized ACC. The dose according to any one of the aspects and embodiments of the present invention refers to the amount of elemental calcium in the ACC. According to one embodiment, the method of the present invention comprises administering less than 150 mg/kg/day or less than 100 mg/kg/day of calcium as stabilized ACC. According to another embodiment, the administered dose of ACC is less than 50 mg/kg/day, less than 30 mg/kg/day, or less than 20 mg/kg/day of calcium as stabilized ACC. According to some embodiments, administration according to the present invention, e.g. oral, sublingual or combined administration comprising administration of 5 to 150, 10 to 120, 15 to 100, 20 to 80, 30 to 70 or 40 to 60 mg/kg/day of calcium as stabilized ACC. According to certain embodiments, administration according to the present invention, e.g. oral, sublingual or combined administration comprising administration of 5 to 80, 10 to 75, 15 to 70, 20 to 65, 25 to 60, 30 to 55, 35 to 50 or 40 to 45 mg/kg/day of calcium as stabilized ACC. According to some embodiment, administration according to the present invention, e.g. oral, sublingual or combined administration comprising administration of about 10 to about 45, about 15 to about 40, about 20 to about 35 mg/kg/day of calcium as stabilized ACC. According to further embodiment, administration according to the present invention, e.g. oral, sublingual or combined administration comprising administration of 0.1 to 30, 0.2 to 28, 0.3 to 26, 0.5 to 24, 1 to 22, 2 to 20, 3 to 18, 3 to 16, 4 to 15, 5 to 14, 6 to 12, or 8 to 10 mg/kg/day of calcium as stabilized ACC. According to some embodiments, administration according to the present invention, e.g. oral, sublingual or combined administration comprising administration of 0.2 to 10, 0.5 to 8, 0.8 to 6, 1 to 5, 1.5 to 4, or 2 to 3 mg/kg/day of calcium as stabilized ACC. According to another embodiment, the administration comprises administration of 500 to 8000 mg/day, 800 to 6000, or 100 to 4000 mg/day of ACC. According to some embodiments, the administration comprises administration of from 200 to 3000 mg/day, from 400 to 2500 or from 600 to 2000 mg/day of calcium as stabilized ACC. According to other embodiments, the administration comprises administration of from 800 to 4000 mg/day, from 1000 to 3000 or from 1500 to 2500 mg/day of calcium as stabilized ACC.

According to some embodiments, the administration e.g. oral, sublingual or combined administration, comprises daily administering of about 600 to about 23,500 mg of ACC. According to one embodiment, the administration comprises administering of 600 to 20,000, 800 to 18,000, 1,000 to 15,000, 1,200 to 12,000, 1,500 to 10,000, and 2000 to 8000 mg/day of ACC. According to some embodiments, the administration comprises administering of 1000 to 12000, 2000 to 11000, 3000 to 10000, 3500 to 9000, 4000 to 8000 mg/day of ACC.

According to some embodiments, the administration comprises administering of 10 to 350 mg/kg/day of stabilized ACC. According to some embodiment, the administration comprises administering of 2 to 100, 3 to 90, 4 to 85, 5 to 80, 10 to 70, or 15 to 60 mg/kg/day of ACC. According to some embodiment, the administration comprises administering of 2 to 10, 3 to 9, 4 to 8.5, 5 to 8 or 6 to 8 mg/kg/day of ACC.

According to some embodiments, the administration comprises administration in a single dose or in multiple separated doses. For example, the daily dose may be separated and administered in 2, 3, 4, 5, 6, 7, 8, 9, 10, 11 or 12 separate doses.

According to some embodiments, the administration is short-term administration, e.g. administration for at least 1, 2, 3, 5, or 7 days. According to other embodiments, the administration is for 1, 2, 3, or 4 weeks. According to further embodiments, the administration is for a long term such as for 1, 2, 3, 4, 5, 6, 7, 8, 9 10, 11, or 12 months. According to other embodiments, the administration for one or more years such as 2, 3, 4, 5, or more years.

According to some embodiments, the use comprises prevention. Therefore, according to some embodiments, the administration is prophylactic. According to some embodiments, the use for preventing myocardial ischemia or infarction is preventive to a subject predisposed to develop the myocardial ischemia or infarction, as described above.

According to some embodiments, the composition for sublingual administration is in the form of a powder of ACC. The powder for sublingual administration is formed of ACC particles.

The term “particles” as used herein refers to a discrete microparticle or a nanoparticle of ACC stabilized by the stabilizer as defined hereinabove, as well as to the aggregates or agglomerates thereof. According to some embodiments, the particles are primary particles of the stabilized ACC. The basic nanoparticles are in the range of 5 to 500 nm or 10 to 300 nm or 20 to 100 nm. In many cases, these nanoparticles immediately agglomerate and aggregate into much larger secondary particles. These aggregation and agglomeration can be then broken by milling and dissolution techniques into smaller particles. According to other embodiments, the particles are agglomerates or aggregates of the primary particles, i.e. secondary particles. The term “particle size” as used herein refers to a measurement of a representative diameter of the secondary particles such as an aggregate or a broken aggregate in at least one dimension.

The particle size of agglomerates can be tailored into ranges by a combination of milling and sieving techniques. In some embodiments, at least 70% of the processed particles of the composition have a particle size of 600 μm or less. In other embodiments, at least 80%, at least 85%, at least 90% or at least 95% of the particles of the composition have a particle size of 600 μm or less. In some embodiments, at least 80%, at least 85%, at least 90% or at least 95% of the particles of the composition have a particle size of 500 um or less. In some embodiments, at least 80%, at least 85%, at least 90% or at least 95% of the particles of the composition have a particle size of 400 μm or less. In some embodiments, at least 80%, at least 85%, at least 90% or at least 95% of the particles of the composition have a particle size of 300 μm or less. In some embodiments, at least 80%, at least 85%, at least 90% or at least 95% of the particles of the composition have a particle size of 200 μm or less. In some embodiments, at least 80%, at least 85%, at least 90% or at least 95% of the particles of the composition have a particle size of 100 um or less. In some embodiments, at least 80%, at least 85%, at least 90% or at least 95% of the particles of the composition have a particle size of 70, 50, 30 μm or less. According to some embodiments, the particle size is from about 20 to about 500 μm, from about 30 to about 450 μm or from about 50 to about 400 μm.

According to any one of the above embodiments, the ACC is a stabilized ACC, i.e. ACC that maintains amorphous for a long period even in humid conditions or in an aqueous environment.

The terms “amorphous calcium carbonate”, “ACC”, “stable ACC” and “stabilized ACC” and “ACC comprising a stabilizer” are used herein interchangeably and refer to the amorphous form of calcium carbonate. The term “stable” as used herein indicates that the calcium carbonate is maintained in the amorphous form for a long period of time, for example for about at least 7 days in the solid form having less than or about 30% crystalline calcium carbonate. According to any one of the above embodiments, the composition is stable for at least 7 days. According to some embodiments, the composition is stable for at least 1 month. According to other embodiments, the composition is stable for at least 3 months. According to a further embodiment, the composition is stable for 6 months. According to certain embodiments, the composition is stable for at least 1 year. According to a particular embodiment, the composition is stable to at least 2 years.

According to any one of the above embodiments, the ACC is stabilized by at least one stabilizer. The term “ACC stabilized” has the meaning of ACC comprising a stabilizer as a part of the ACC particle. The terms “stabilizing agent” and “stabilizer” are used herein interchangeably and refer to any molecule, ion or substance that contributes to preserving calcium carbonate in the amorphous state during ACC production, formulating and/or storage. According to the teachings of the present invention, the ACC acts as an active agent conferring improvement in athletic and muscle performance. Any ACC that remains stable may be used according to the teaching of the present invention. Any compound that may stabilize ACC in its amorphous form is suitable for the implementation of the present invention. The terms “stable ACC” and “stabilized ACC” are used herein interchangeably and indicates that calcium carbonate is maintained in its amorphous form for a prolonged period of time having less than or about 30% of conversion to a crystalline form. According to some embodiments, the term refers to the solid stabilized ACC as well as to stabilized ACC dispersed in a liquid carrier such as aqueous (e.g. water) or non-aqueous liquid carrier.

In certain embodiments, the stabilizing agent is a single agent. In other embodiments, the use of several stabilizing agents is encompassed. In some cases, the stabilizers are present within the molecular matrix of the ACC particles. In some cases, they are deposited externally and in other cases, they are both internal or external. The internal stabilizers or combination of stabilizers can be the same or different than the external ones.

### ACC Stabilizers

The stabilizer may comprise a molecule having one or more functional groups selected from, but not limited to, hydroxyl, carboxyl, ester, amine, phosphino, phosphono, phosphate, sulfonyl, sulfate or sulfino groups. The hydroxy bearing compounds, combined with the hydroxide, optionally also bear other functions like carboxyl, etc. but with the hydroxyl not being esterified.

According to some embodiments, the stabilizer has low toxicity or no toxicity to mammalian cells or organism, and in particular to a human being. According to some embodiment, the stabilizer is of food, nutraceutical or pharmaceutical grade.

In certain embodiments, the ACC stabilizing agent is independently at each occurrence, an organic acid, phosphorylated, phosphonated, sulfated or sulfonated organic compound, phosphoric or sulfuric ester of a hydroxyl carboxylic acid, an organoamine compound, an organic compound comprising a hydroxyl, an organophosphorous compound or a salt thereof, phosphorylated amino acids and derivatives thereof, a bisphosphonate compound, an organophosphate compound, an organophosphonate compound, an inorganic phosphorous acid, an organic compound having multiple functional groups as defined above, an inorganic phosphate and polyphosphate compound, an organic compound having a polyphosphate chain, an organic surfactant, a bio-essential inorganic ion, or any combination thereof.

According to some embodiments, the stabilizer is an organic acid. According to certain embodiments, the organic acid is selected from ascorbic acid, citric acid, lactic acid, acetic acid, oxalic acid, malonic acid, glutaconic acid, succinic acid, maleic acid, lactic acid, aconitic acid, and optionally include compounds having at least two carboxylic groups and molecular weight not larger than 250 g/mol, such as citric acid, tartaric acid, malic acid, etc. According to one particular embodiment, the stabilizer is citric acid.

In another embodiment, the phosphoric ester of hydroxyl carboxylic acids is a phosphoenolpyruvate. In another embodiment, the phosphoric or sulfuric esters of hydroxyl carboxylic acids comprise amino acids. Examples of such esters are phosphoserine, phosphothreonine, sulfoserine, sulfothreonine and phosphocreatine.

The hydroxyl bearing compounds combined with hydroxide may comprise, for example, mono-, di-tri-, oligo-, and polysaccharides like sucrose or other polyols like glycerol. The hydroxyl bearing compounds may further comprise hydroxy acids like citric acid, tartaric acid, malic acid, etc., or hydroxyl-bearing amino acids such as serine or threonine. Each possibility represents a separate embodiment, of the present invention.

Some specific unlimited examples for such ACC stabilizers that include phytic acid, citric acid, sodium pyrophosphate dibasic, adenosine 5′-monophosphate (AMP) sodium salt, adenosine 5′-diphosphate (ADP) sodium salt and adenosine 5′-triphosphate (ATP) disodium salt hydrate, phosphoserine, phosphorylated amino acids, food grade surfactants, sodium stearoyl lactylate, and combinations thereof.

According to some embodiments, the stabilizer comprises at least one component selected from phosphoric or sulfuric esters of hydroxyl carboxylic acids, such as phosphoenolpyruvate, phosphoserine, phosphothreonine, sulfoserine or sulfothreonine and hydroxyl bearing organic compounds, selected from mono-, di-, tri-, oligo-and polysaccharides, for example, sucrose, mannose, glucose.

The hydroxyl bearing compound may further comprise at least one alkali hydroxide, such as sodium hydroxide, potassium hydroxide and the like. The phosphorylated acids may be present in oligopeptides and polypeptides. In other embodiments, of the invention, the stabilizer is an organic acid selected from monocarboxylic acid or multiple carboxylic acid, e.g. dicarboxylic acid or tricarboxylic acid. Each possibility represents a separate embodiment, of the invention. The organic acid may be as defined above.

In some embodiments, of the invention, the ACC stabilizer is selected from phosphorylated amino acids, polyols and combinations thereof. In some embodiments, the stable ACC comprises a phosphorylated compound as a stabilizer wherein the phosphorylation is performed on the hydroxyl group of an organic compound. In some embodiments, the stable ACC comprises a stabilizer selected from the group consisting of citric acid, phosphoserine, phosphothreonine and combinations thereof. The non-limiting examples of stabilizers containing phosphate, phosphite, phosphonate groups and salts or esters thereof include phytic acid, dimethyl phosphate, trimethyl phosphate, sodium pyrophosphate, tetraethyl pyrophosphate, ribulose bisphosphate, etidronic acid and other medical bisphosphonates, 3-phosphoglyceric acid salt, glyceraldehyde 3-phosphate, 1-deoxy-D-xylulose-5-phosphate sodium salt, diethylene triamine pentakis(methylphosphonic acid), nitrilotri(methylphosphonic acid), 5-phospho-D-ribose 1-diphosphate pentasodium salt, adenosine 5′-diphosphate sodium salt, adenosine 5′-triphosphate disodium salt hydrate, α-D-galactosamine 1-phosphate, 2-phospho-L-ascorbic acid trisodium salt, α-D-galactose 1-phosphate dipotassium salt pentahydrate, α-D-galactosamine 1-phosphate, O-phosphorylethanolamine, disodium salt hydrate, 2,3-diphospho-D-glyceric acid pentasodium salt, phospho(enol)pyruvic acid monosodium salt hydrate, D-glyceraldehyde 3-phosphate, sn-glycerol 3-phosphate lithium salt, D-(-)-3-phosphoglyceric acid disodium salt, D-glucose 6-phosphate sodium salt, phosphatidic acid, ibandronate sodium salt, phosphonoacetic acid, DL-2-amino-3-phosphonopropionic acid or combinations thereof.

In some embodiments, the stabilizers can be bio-essential inorganic ions including, inter alia, Na, K, Mg, Zn, Fe, P, S, N, P, or S in the phase of oxides, or N as ammonia or nitro groups.

The stabilized ACC may be stabilized by more than one stabilizer, e.g. 2, 3, or more stabilizers. The stabilizers can be added during the synthesis and precipitation of the ACC primary particles and they are defined as “internal stabilizer”. Stabilizers can be added after the synthesis and bind to the external surface of the particles. They are defined as “external stabilizers”. In some embodiments, where both internal and external stabilizers are used the internal stabilizer and the external stabilizer are similar. In other embodiments, the internal stabilizer and the external stabilizer are different stabilizers. The internal and the external stabilizers may be each independently as defined hereinabove and each can be a combination of more than one type of stabilizer.

The stable ACC can comprise more than two stabilizers, wherein one or more stabilizers are added to the ACC during the formation and precipitation of the ACC.

According to some embodiments, the at least one stabilizer is selected from the group consisting of a polyphosphate, bisphosphonate, phosphorylated amino acid, citric acid, and any combination thereof. In some embodiments, more than one stabilizer, e.g. 2, 3, or 4 stabilizers are added.

According to one embodiment, ACC is stabilized by a combination of phosphoserine and citric acid. According to another embodiment, ACC is stabilized by a combination of triphosphate and citric acid.

According to some embodiments, the stabilizer is a polyphosphate or pharmaceutically acceptable salts thereof. According to some embodiments, the polyphosphate is physiologically compatible, water-soluble polyphosphate salt selected from the group consisting of sodium, potassium, and any other essential cation of polyphosphate. In one embodiment, the polyphosphate is organic or inorganic polyphosphate. The term “polyphosphate” as used herein refers to polymeric esters of PO4. According to some embodiments, the polyphosphate is a physiologically compatible water-soluble polyphosphate salt selected from the group consisting of sodium and potassium polyphosphate. In some embodiments, the polyphosphate is an inorganic polyphosphate or pharmaceutically acceptable salts thereof. Not-limiting examples of such salt are Na, K, Mg, Mn, and Zn. According to some embodiments, the inorganic (poly)phosphate comprises 2 to 10 phosphate groups, e.g. 2, 3, 4, 5, 6, 7, 8, 9, or 10 phosphate groups. According to some embodiments, the inorganic polyphosphate is selected from pyrophosphate, triphosphate, and hexametaphosphate. According to one embodiment, the stabilizer is pyrophosphate or pharmaceutically acceptable salts thereof such as sodium pyrophosphate. According to another embodiment, the stabilizer is an inorganic triphosphate or pharmaceutically acceptable salts thereof such as sodium triphosphate. The term “triphosphate” and “tripolyphosphate” are used herein interchangeably. According to a further embodiment, the stabilizer is hexametaphosphate or a pharmaceutically acceptable salt thereof such as sodium hexametaphosphate.

According to some embodiments, the stabilizer is a bisphosphonate or pharmaceutically acceptable salts thereof. The non-limiting examples of salt are Na, K, Mg, Mn and Zn.

The term “bisphosphonate” as used herein refers to organic compounds having two phosphonate (PO(OH)₂) groups. The term further relates to compounds having a backbone of PO₃-organic-PO₃. Most typical is a series of bisphosphonates that are used as pharmaceuticals for treating osteoporosis. According to some embodiments, the bisphosphonate is selected from the group consisting of etidronic acid, zoledronic acid, medronic acid, alendronic acid, and a pharmaceutically acceptable salt thereof. According to some embodiments, the stabilizer is an etidronic acid or a pharmaceutically acceptable salt thereof. According to another embodiment, the stabilizer is a zoledronic acid or a pharmaceutically acceptable salt thereof. According to a further embodiment, the stabilizer is a medronic acid or a pharmaceutically acceptable salt thereof. According to certain embodiments, the stabilizer is alendronic acid or a pharmaceutically acceptable salt thereof.

According to certain embodiments, the stabilizer is a phosphorylated amino acid. According to one embodiment, the phosphorylated amino acid is phosphoserine. According to another embodiment, the phosphorylated amino acid is phosphothreonine.

According to some embodiments, the ACC composition comprises a combination of the stabilizers disclosed above.

According to some embodiments, the stabilizer is an inorganic polyphosphate or a bisphosphonate as defined hereinabove, and the molar ratio between P atoms of the stabilizer and Ca atoms of the ACC (P:Ca molar ratio) is about 1:90 to 1:1. In one embodiment, the P:Ca molar ratio is about 1:40 to about 1:1. In a further embodiment, the P:Ca molar ratio is about 1:35 to about 1:2. In certain embodiments, the P:Ca molar ratio is about 1:30 to about 1:3. In certain embodiments, the P:Ca molar ratio is about 1:28 to about 1:3. In other embodiments, the P:Ca molar ratio is about 1:25 to about 1:4. In further embodiment, the P:Ca molar ratio is about 1:20 to about 1:5. In another embodiment, the P:Ca molar ratio is about 1:20 to about 1:6. In a particular embodiment, the P:Ca molar ratio is about 1:15 to about 1:5. In another particular embodiment, the P:Ca molar ratio is about 1:25 to about 1:5. According to some embodiments, such inorganic polyphosphate is pyrophosphate, triphosphate, hexametaphosphate or a pharmaceutically acceptable salt thereof. According to another embodiment, the bisphosphonate is alendronic acid, etidronic acid, zoledronic acid or medronic acid and the P:Ca molar ratio is as defined hereinabove.

According to some embodiments, the calcium content (Ca content) of such compositions comprising stabilizers is about 1 wt % to about 39 wt %, about 5 wt % to about 39 wt %, about 10% to about 39 wt %, about 15% to about 39 wt %, about 20 wt % to about 38 wt %, about 25 wt % to about 38 wt %, or about 30 wt % to about 38 wt % of the dry ACC particles The terms “Ca content” and “calcium content” is used herein interchangeably and refer to the content of calcium of the ACC in the final composition.

In certain embodiments, the P:Ca molar ratio is about 1:40 to about 1:1, and the Ca content is about 20 wt % to about 39 wt %. In some embodiments, the molar ratio is 1:28 to about 1:3, and the Ca content is about 30 wt % to about 38 wt % of the dry ACC particles. In another embodiment, the molar ratio is 1:25 to about 1:5, and the Ca content is about 30 wt % to about 36 wt % of the dry ACC particles.

According to some embodiments, the stabilized ACC powder comprises absorbed and adsorbed water, from about 1 wt % to about 18 wt %, from about 4 wt % to about 15 wt %, and from about 6 wt % to about 10 wt %. According to some embodiments, the stabilizer is polyphosphate or bisphosphonate and the molar ratio between P atoms of the stabilizer and Ca atoms of the ACC is about 1:90 to 1:1.

According to some embodiments, the stabilizer is selected from the group consisting of a polyphosphate, phosphorylated amino acid, bisphosphonate, citric acid, tartaric acid and any combination thereof. According to one embodiments, the polyphosphate is selected from the group consisting of triphosphate, pyrophosphate, and hexametaphosphate, the phosphorylated amino acid is phosphoserine or phosphothreonine, and the bisphosphonate is selected from the group consisting of alendronate, etidronic acid, zoledronic acid and medronic acid. According to some embodiments, the polyphosphate is an inorganic polyphosphate.

According to one embodiment, the stabilizer is selected from the group consisting of organic acids, phosphorylated, phosphonated, sulfated or sulfonated organic compound, phosphoric or sulfuric esters of hydroxy carboxylic acids, phosphorylated amino acids, bisphosphonate, organic polyphosphate, hydroxyl bearing organic compounds, derivatives thereof, proteins and any combinations thereof.

According to another embodiment, the stabilizer is selected from the group consisting of phosphoserine, adenosine triphosphate, adenosine diphosphate, phytic acid, citric acid, etidronic acid, pyrophosphate, polyphosphate, inorganic triphosphate, hexamethaphosphate, ethanol, and any combination thereof.

In most cases, the ACC contains 1 to 20 wt % and preferably no more than 10 wt % of adsorbed water and maintain its stabilization in the presence of a stabilizer and further storage in dry conditions. ACC powder that was used in the examples contained about 6 to 10 wt % of water when formulated. In terms of calcium content, it means that the calcium content in ACC is in a practical range of 28 to 38 wt % of its composition. For practical calculation, the average calcium content is defined as 30 wt % in this application.

The term “pharmaceutical composition” as used herein refers to any composition comprising at least stabilized ACC, and optionally at least one additional pharmaceutically acceptable carriers, stabilizers, and/or bulking agents.

According to any above aspects and embodiments, the composition of the present invention is a food supplement. According to any above aspects and embodiments, the composition of the present invention is a pharmaceutical composition.

Formulations of the composition of the present invention may be adjusted according to the required applications. In particular, the composition may be formulated using a method known in the art to provide a rapid, continuous or delayed release of the active ingredient after administration to mammals. For example, the formulation may be any one selected from plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules. According to some embodiments, the composition is a powder. According to certain embodiments, the composition is a powder for a sublingual administration. According to other embodiments, the composition is in the form of a tablet or capsules for oral administration. According to some embodiments, the composition for oral administration may be an enteric-coated composition or an enteric capsule.

Formulations can also include excipients for aiding the manufacturing, storage, and efficiency of the administration. Examples are silicon dioxide and microcellulose as anticaking agents, magnesium stearate as a lubricant, and sucralose, mannitol, sorbitol, erythritol, menthol and citric acid as flavoring agents.

According to some embodiments, the composition is a nutraceutical composition. As used herein, the term “nutraceutical composition” refers to a composition suitable for use in human beings or animals, comprising one or more natural products with therapeutic action which provide a health benefit or have been associated with disease prevention or reduction.

The term “food supplement” is used to mean a product containing said composition and intended to supplement the food by providing nutrients that are beneficial to health according to any acceptable directive, such as European directive. For example, a food supplement may be a capsule or a tablet for swallowing, or powder or small vial to mix with food and providing beneficial health effects. The food supplement may be also formulated as a sublingual composition. The food supplement may comprise aside the active agent edible carrier and/or excipients. According to some embodiments, the edible carrier and/or excipient is a pharmaceutically acceptable carrier and/or excipient.

The term “edible carrier” refers to compounds, materials, compositions, and/or dosage forms that are suitable for use in contact with the tissues of a subject. Each carrier must also be “acceptable” in the sense of being compatible with the other ingredients of the formulation. The term “edible carrier” as used herein means a material that can be administered, consumed, digested or passed through the digestive system of an animal or human without any toxic effect. These edible carrier materials can exist as either a solid or liquid at room temperature.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, and the like, that are compatible with pharmaceutical administration. The use of such media and agents for pharmaceutically active substances is well known in the art. The compositions may contain other active compounds providing supplemental, additional, or enhanced therapeutic functions.

The terms “pharmaceutically acceptable” and “pharmacologically acceptable” include molecular entities and compositions that do not produce an adverse, allergic, or other untoward reactions when administered to an animal, or human, as appropriate.

According to any one of the above embodiments, the composition of the present invention, is formulated in any known form such as powder, suspension, tablet, or capsule.

Having now generally described the invention, the same will be more readily understood through reference to the following examples, which are provided by way of illustration and are not intended to be limiting of the present invention.

### EXAMPLES

### Example 1

Model—mouse model of myocardial infraction (MI).

C57/b7 weeks old male mice were used.

Three treatments groups were treated as follows (the number of mice refers to the final amount used for calculation):
1. IP administration of a solution comprising stabilized ACC immediately after induction of MI for 28 days (6 mice, denoted as Treatment After)
2. IP administration of a solution comprising stabilized ACC 7 days before induction of MI and for additional 28 days after MI induction (5 mice, denoted as Treatment before)
3. IP administration of saline immediately after induction of MI and continuing treatment with saline for 28 days. (8 mice, denoted as Control)

Stabilized ACC: ACC was stabilized using inorganic triphosphate (8-12%, optionally with 1% of citric acid. The solution contains 1% w/v of stabilized ACC (0.45% (w/v) elemental calcium), each mouse was injected with 0.2 ml (0.01 ml/gr) of the stabilized ACC. Alternatively, ACC is stabilized with phosphoserine, polyphosphate comprising 2-10 phosphate groups, bisphosphonates, organic acid, phytic acid citric acid, and any combination thereof. Exemplary method of preparation of stabilized ACC is described below.

Preparation of 10% TP-1% citric acid ACC (ACC stabilized with 10% inorganic triphosphate and 1% citric acid) formulated as cell culture medium supplement.

36 ml of 3% Calcium chloride solution were mixed with 4 ml of 0.27% Citric Acid solution and with 10 ml of 0.5406% Triphosphate solution. Then 40 ml of 1.9485% Sodium carbonate solution was added to precipitate ACC. 10 ml of the stabilizing solution containing 0.5406% triphosphate was added to the ACC suspension creating stabilized ACC suspension. The obtained suspension was used. Alternatively, the suspension was filtered using a Buchner funnel, the cake was washed with water and the cake was further dried, e.g. in the oven. In alternative methods the ACC is stabilized using inorganic polyphosphate having 2-10 phosphates, phosphoserine, bisphosphonates, organic acid and any combination thereof Further examples for stable ACC and the preparation thereof may be found in International Patent Applications Nos. WO 2009/053967, WO 2014/024191 and WO 2016/193982.

MI Procedure The animals were acclimatized for 5 days, they were kept in a 12 hours day/night regimen with food and water ad libitum. On the day of MI induction mice were anesthetized with 4% isoflurane and connected to assisting respiration machine. During the surgery, 8-0 surgical thread was used to ligate the left anterior descending artery (LAD). Immediately after the ligation of the LAD, ischemic tissue can be seen. After the surgical procedure, the mice were returned to cage.

At the end of the experiment (28 days after MI induction) the animals were euthanized and hearts were taken for histopathology evaluations with Masson's trichrome staining.

Echocardiograph evaluations were performed before the surgery (day 0) and at days 2 and 28 to all mice.

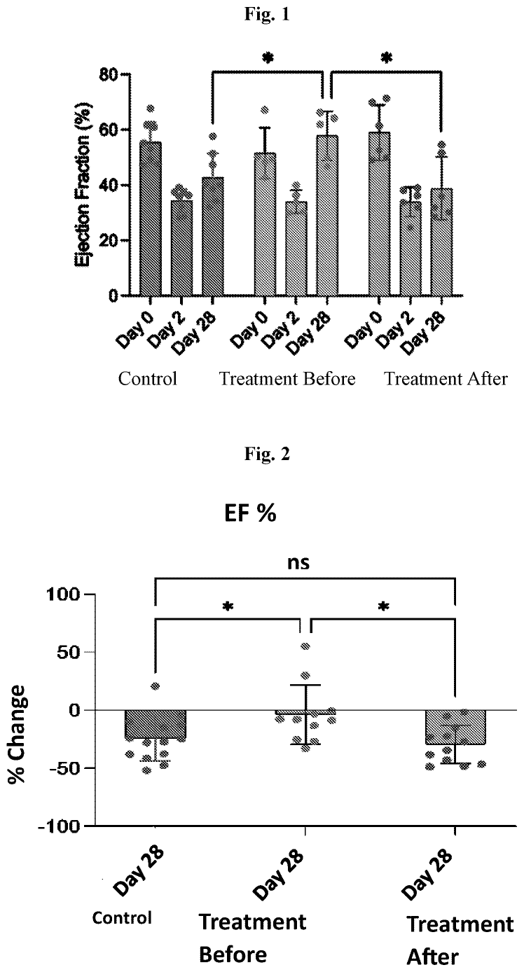

Results are presented in FIGS. 1 and 2. FIG. 1 shows the ejection fraction (the percentage of blood that is pumped out of the left ventricle in each heart bit) of the different treatment. The results show that only the group that was treated before MI induction was able to restore the ejection fraction levels similar to those seen before damage was induced (p<0.05). FIG. 2 shows the percentage of the change in the Ejection Fraction of each treatment group on day 28 compared to baseline (day 0). The figure clearly shows that the preventive treatment with stabilized ACC has enabled the mice to heal faster compared to other treatments (p<0.05), showing almost complete recovery with no change compared to baseline in the Ejection Fraction after 28 days.

### Example 2—The Effect of Stabilized ACC Compared to Calcium Chloride on Cardiomyocytes and Endothelial Cell In-Vitro

In this study cardiomyocytes and endothelial cells were obtained from 3 days old ICR mice (males and females). Heart slices from 3-day-old mice were minced and enzymatically (using collagenase) separated and plated in a culture dish. A general protocol can be found in Ehler E, Moore-Morris T, Lange S. Isolation and culture of neonatal mouse cardiomyocytes. J Vis Exp. 2013; (79): 50154. Cells were cultured in 24 well plates. Calcium depleted DMEM/F-12 (Ca-depleted) medium was used. 1 mM and 2 mM ACC or CaCl₂ were added to Ca-depleted medium. Cells were cultured for 4 days in these different conditions in triplicates. At the end of the culture period, the cells were stained with cTnT (stains Cardiac Troponin T) which is a marker for cardiomyocytes and with Ki67 stain which stains proliferative cells. Cells were examined with a fluorescent microscope. Cells that showed both stains (green for cTnT and red for Ki67) were proliferating cardiomyocytes. Many pictures were analyzed and quantified.

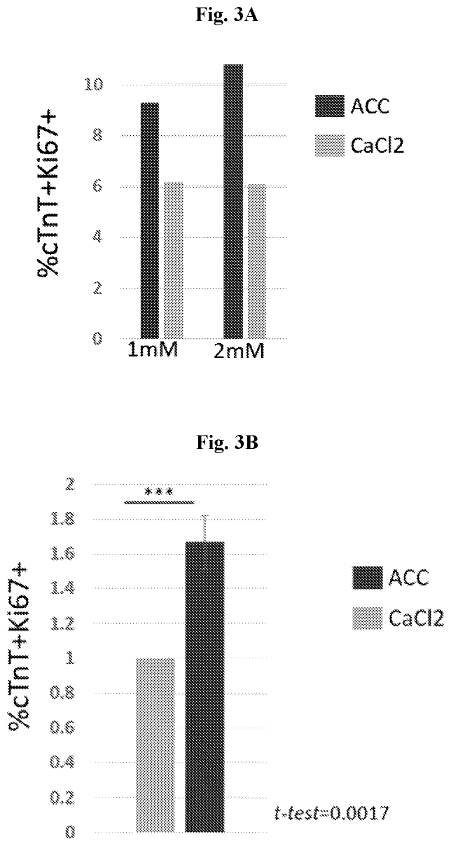

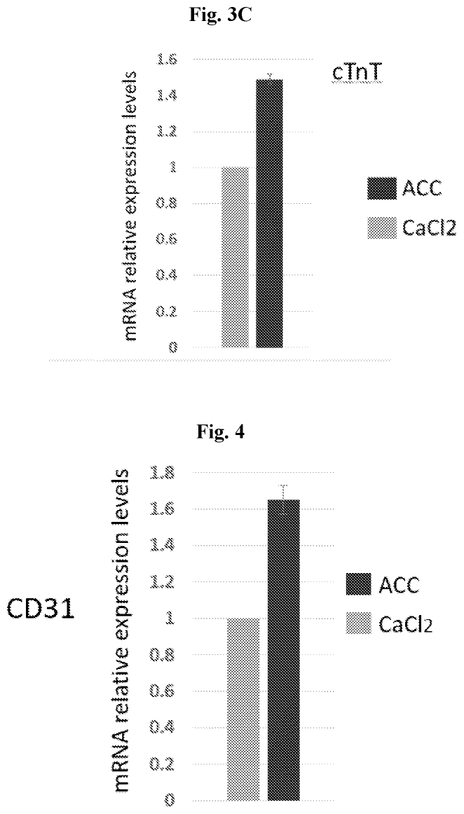

Results are presented in FIGS. 3A-3C and show that ACC caused an increased cardiomyocytes proliferation compared to CaCl₂ in both concentrations. As can be seen from FIG. 3B showing a proliferation of cardiomyocytes in the presence of ACC relative to proliferation in the presence of CaCl₂ (same concentration of elemental calcium), there is a significant increase in cTnT, which is a marker for cardiomyocyte proliferation. This can be also seen on the mRNA level (FIG. 3C) In addition, ACC had a beneficial effect on cardiomyocytes contraction (as was seen in video, data not shown) compared to CaCl₂ showing much more spontaneously contracting cells in those cultured with ACC compared to CaCl₂.

Endothelial cells were also evaluated using CD31 marker and they were quantified by evaluating relative mRNA expression using RT-PCR. The results are presented in FIG. 4. It can be seen that in the presence of ACC (2mM) ACC cultured, endothelial cells (expressing CD31) were found to be more abundant than in the presence of corresponding concentration (referring to calcium) of CaCl₂.

These results suggest that ACC improves the functionality and performances of cardiomyocytes and endothelial cells which may indicate also the improved repair mechanisms. It is well established that interaction between endothelial cells and cardiomyocytes regulates early cardiac development and adult cardiomyocyte function. This includes the effect on the contractile state. Cardiomyocytes depend on endothelial cells not only for oxygenated blood supply but also for local protective signals that promote cardiomyocyte organization and survival [Hsieh P C, Davis M E, Lisowski L K, Lee RT. Endothelial-cardiomyocyte interactions in cardiac development and repair. Annu Rev Physiol. 2006; 68:5 1-66].

### Example 3-ACC Effect on the Regeneration of New Blood Vessels in Mice That Underwent MI

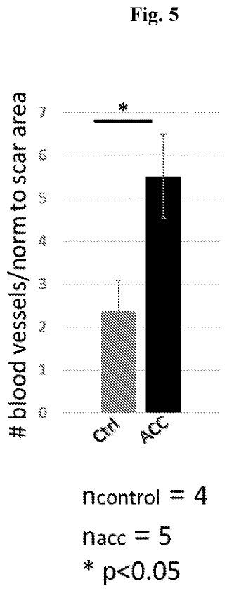

Heart tissue from the damaged area (i.e. scar tissue) of the mice from experiment 1 above, either from the group treated with ACC 1 week prior to induction of MI and continued for addition 28 days until mice were euthanized, or from control group that received saline after MI induction for 28 days until being euthanized, were used for histopathological evaluations. The following stains were performed: cTnT (for cardiac troponin T), α-SMA (stains smooth muscle actin alpha) and DAPI (stains dead cell nuclei) were performed on slides obtains from these 2 treatment groups. Analysis of the ratio between new blood vessels in the scar area (seen by α-SMA stain) compared to the area of the damaged tissue (cTnT and DAPI stains) was done. FIG. 5 show the results.

The results in FIG. 5 show that mice received ACC 1 week prior MI induction and continued to receive ACC for additional 28 days had significantly more new blood vessels compared to control mice that received only saline/vehicle for 28 days after MI induction. These results demonstrate a protective effect of ACC minimizing damaged caused by MI as well as improved healing process as seen by the new blood vessels generated.

Although the present invention has been described herein above by way of preferred embodiments thereof, it can be modified, without departing from the spirit and nature of the subject invention as defined in the appended claims.

1-12. (canceled)

13. A method for treating or preventing myocardial ischemia in a subject in need thereof, the method comprising:
administering to said subject a composition comprising amorphous calcium carbonate (ACC) stabilized by at least one stabilizer, thereby treating or preventing myocardial ischemia.

14. The method according to claim 13, wherein the myocardial ischemia is caused by myocardial infarction.

15. The method according to claim 13, wherein the composition is administered in a mode selected from sublingual, inhalation, IV, oral administration or by a combination of administration selected from oral and sublingual, inhalation and sublingual, oral and inhalation, and oral, inhalation and sublingual administrations.

16. The method according to claim 13, wherein administering to said subject a composition comprising ACC stabilized by at least one stabilizer includes administering from about 5 to about 200 mg/kg/day of the stabilized ACC.

17. The method according to claim 13, wherein the administering comprises administration of 1, 2, 3, 4, 5, 6, 7, 8, 9, 10, 11 or 12 separate doses per day.

18. The method according to claim 15, wherein the composition for sublingual administration is a powder composition comprising secondary particles of ACC having a size of below 500 μm.

19. The method according to claim 13, wherein the treatment comprises enhanced angiogenesis.

20. The method according to claim 13, wherein administering to said subject a composition comprising ACC stabilized by at least one stabilizer includes administering the composition to the subject that is predisposed to develop the myocardial ischemia.

21. The method according to claim 20, wherein the subject is selected from a subject suffering from diabetes, high blood pressure, high blood cholesterol level, high blood triglyceride level, obesity, having genetic predisposition, stress, has a history of heart disease of MI or smoking subjects.

22. The method according to claim 13, wherein the stabilizer is selected from the group consisting of inorganic polyphosphates, organic acids, phosphorylated amino acids, phosphorylated, phosphonated, sulfated or sulfonated organic compounds, phosphoric or sulfuric esters of hydroxy carboxylic acids, bisphosphonates, organic polyphosphates, hydroxyl bearing organic compounds, derivatives thereof, proteins, and any combinations thereof.

23. The method according to claim 22, wherein the stabilizer is selected from the group consisting of inorganic triphosphate or a salt thereof, phosphoserine, citric acid, sodium triphosphate and citric acid, adenosine triphosphate, adenosine diphosphate, phytic acid, etidronic acid, pyrophosphate, polyphosphate, hexamethaphosphate, ethanol, and any combination thereof.
